(19) United States
(12) Patent Application Publication
(10) Pub. No.: US 2020/0405447 A1
(43) Pub. Date: Dec. 31, 2020

(54) METHOD FOR MONITORING THE POSITION OF TEETH

(71) Applicant: DENTAL MONITORING, Paris (FR)

(72) Inventors: Philippe SALAH, Paris (FR); Emmanuel SALAH, Aulnay Sous Bois (FR); William AYACHE, Neuilly sur Seine (FR)

(73) Assignee: DENTAL MONITORING, Paris (FR)

(21) Appl. No.: 17/016,653

(22) Filed: Sep. 10, 2020

Related U.S. Application Data
(63) Continuation-in-part of application No. 15/023,537, filed on Mar. 21, 2016, now Pat. No. 10,799,321.

(30) Foreign Application Priority Data
Sep. 19, 2013 (FR) 13 59038

(51) Int. Cl. A61C 7/00; A61C 9/00; A61C 13/34; A61B 5/00; A61B 1/24; G06T 17/00

(52) U.S. Cl. CPC A61C 7/002; A61C 9/0053; A61C 13/34; G06T 2210/41; A61B 5/6898; A61B 1/24; G06T 17/00; A61B 5/0013

(57) ABSTRACT

A method for monitoring the position of a patient's teeth includes the following steps: a) modeling a target position of the teeth in the form of a target model; b) after a time interval, modeling an updated position of the teeth in the form of an updated model; and c) comparing the target and updated models. The updated model is created by the patient himself or by one of his relations.

The present application is a continuation-in-part application of U.S. patent application Ser. No. 15/023,537 filed Mar. 21, 2016, which in turn is a U.S. national stage application of PCT/IB2014/064658 filed Sep. 19, 2014. Each of the prior applications is incorporated by reference herein in its entirety.

### TECHNICAL FIELD

The present invention relates to a method for monitoring the position of a patient's teeth, and to a computer program for implementing this method.

### PRIOR ART

After an orthodontic treatment, it is necessary for the treated patient to have his teeth monitored on a regular basis, especially in order to check that the position of the teeth has not developed unfavorably. This unfavorable development is also called a “relapse”. Traditionally, the patient therefore visits his orthodontist at regular intervals in order to have these checks carried out. He can also visit his dentist, who is also able to detect any imperfection in the position of the teeth.

Many patients fail to attend these checkups, which would allow any relapse situations to be detected. The teeth are therefore able to readopt a position of malocclusion which, in order to be corrected, requires new orthodontic treatment, and the latter may be as extensive as the initial treatment.

In addition, the visits are inconvenient for the patient and place stress on the orthodontist.

An object of the present invention is to solve, at least in part, the aforementioned problems.

### SUMMARY OF THE INVENTION

The invention makes available a method for monitoring the position of a patient's teeth, said method comprising the following steps:
a) modeling a target position of said teeth in the form of a target model;
b) after a time interval, modeling an updated position of said teeth in the form of an updated model;
c) comparing said target and updated models.

A method according to the invention may also include one or more of the following optional features:
the target model is a three-dimensional model;
the target model is a model of the position of the patient's teeth after an orthodontic treatment;
the target model corresponds to an actual position of the teeth, that is to say it is not a theoretical model corresponding to a future position at the time when step a) is carried out;
the target model supplies information concerning the position of the teeth with an error of less than 5/10 mm, preferably less than 3/10 mm, preferably less than 1/10 mm;
the time interval is longer than 1 month and/or is determined by the patient or, preferably, by an orthodontist;
step b) is carried out at a distance from step a), that is to say at a different location from the one in which step a) is carried out, in particular more than 50 m, more than 100 m or more than 1 km from the location where step a) is carried out, in particular outside the orthodontic practice;
step b) is not carried out in a dental practice, an orthodontic practice or an orthodontic laboratory;
use is made of an individual apparatus chosen from the group comprising a cell phone, a “connected” photographic apparatus, a smart watch, a digital tablet, a portable 3D scanner, and a computer linked to an image acquisition system, such as a webcam or a digital photographic apparatus, in order to implement step b) and/or step c), preferably step b) and step c);
the updated model comprises a video or a photo of the patient's teeth, preferably a photo of the patient from the front, a photo taken from the patient's right and a photo taken from the patient's left;
the updated model comprises a video of the patient's teeth;
step b) is preferably carried out by the patient or a relation of the patient, but it may be carried out by a dentist;
at step b), one or more photos and/or videos of the patient's teeth are processed in order to create a three-dimensional updated model;
prior to step c), preferably prior to step b), the patient or a dental professional loads the target model onto said individual apparatus or makes it available for loading onto said individual apparatus; the patient preferably downloads the target model from the Internet;
information is generated at step c), the content of which information depends on the difference or “distance” between the target model and updated model, and this information is preferably transmitted to said patient and/or to a dental professional;
depending on the comparison at step c), the patient and/or a dental professional is informed of the probable need for a consultation with a dentist or an orthodontist;
step c) concerning comparison of the models is carried out either on a personal apparatus of the patient's, or with an app used by a dental professional, or with a dedicated third-party server.

The invention also relates to the use of a method according to the invention in order to
detect a relapse, and/or
determine a speed of change in the position of the teeth, and/or
optimize the scheduling of visits to an orthodontist or a dentist, and/or
evaluate the efficacy of an orthodontic treatment, and/or
evaluate the change of position of teeth toward a theoretical model corresponding to a defined position of the teeth, in particular an improved position of the teeth.

The invention also relates to a computer program and in particular to a specialized app configured for implementation of step b) and/or step c), preferably of both steps b) and c), a computer medium on which such a computer program is stored, for example a memory or a CD-ROM, and a personal apparatus, in particular a cell phone or a tablet, on which such a program is loaded.

The personal apparatus may in particular comprise an acquisition module and/or a processing module and/or a comparison module and/or a communication module having one or more of the features described below, so as to be able to execute one or more of the operations of a method according to the invention.

The invention also relates to a system comprising
a dental professional,
a patient,

said professional being equipped with an apparatus that is able to implement step a) of a method according to the invention, preferably in order to create a three-dimensional model of the dentition of said patient,

said patient being equipped with a personal apparatus, preferably a cell phone, on which a specialized app is loaded that is able to carry out steps b) and c) of a method according to the invention.

The professional can in particular be equipped with a three-dimensional scanner.

The invention relates, finally, to a method for therapeutic or cosmetic treatment comprising the following steps:
A. modifying the position of a patient's teeth by means of an active retainer apparatus, for example an aligner of the Invisalign® type, or an apparatus comprising an arch and brackets;
B. at the end of step A, that is to say after the position of the teeth has been corrected and the patient is no longer wearing an active retainer apparatus, checking for a relapse by means of a method for monitoring the position of the teeth according to the invention.

### Definitions

A “patient” is understood as any person on whom a method is carried out in order to monitor the teeth, whether or not this person is ill.

A “dental professional” is understood as a dentist, an orthodontist or an orthodontic laboratory.

A “dentist” is understood as a dentist or a dental assistant working under the responsibility of a dentist.

A “dental site” is understood as a building in which dental and/or orthodontic activities occur, for instance a dentist office, an orthodontic office, an orthodontic laboratory, or a data center in which dental or orthodontic data are treated to establish a diagnosis or to design an orthodontic appliance for a plurality of patients, for instance for more than 300 patients.

A “cell phone” is an apparatus of less than 500 g, equipped with a sensor allowing it to record images, capable of exchanging data with another apparatus more than 500 km from the cell phone, and capable of displaying said data, especially said images.

A “tablet” is a portable computer with a touch-sensitive screen.

A 3D scanner is an apparatus with which it is possible to obtain a three-dimensional representation of an object.

The terms “comprising a” or “having a” are understood as meaning “having at least one”, unless otherwise indicated.

### BRIEF DESCRIPTION OF THE FIGURES

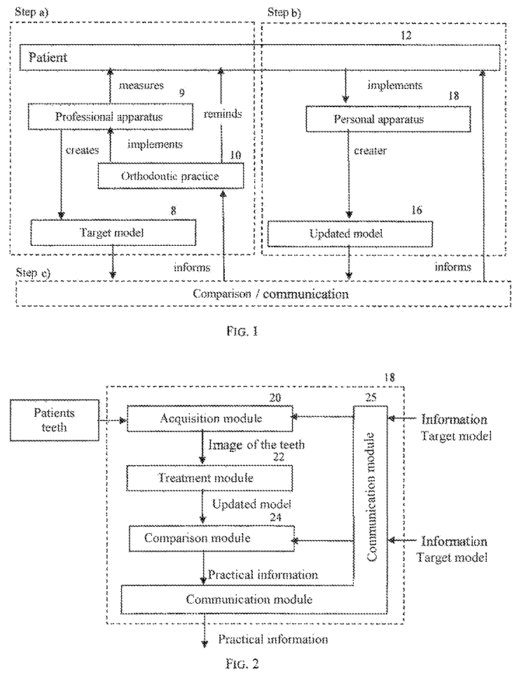

Other features and advantages of the invention will become clearer on reading the following detailed description and on studying the attached drawing, in which FIG. 1 and FIG. 2 illustrate, respectively, a method and the contents of a cell phone that may be used for carrying out a method according to the invention.

### DETAILED DESCRIPTION

A method according to the invention has the steps a) to c) mentioned above.

Without limiting the scope of the invention, the method described below is implemented in order to monitor a relapse after an orthodontic treatment.

At step a), a target model 8 of the patient's teeth is created (see FIG. 1).

In the context of monitoring a relapse, the target model is a model of the teeth in their corrected position, resulting from the implementation of the orthodontic treatment. The target model is thus created after the orthodontic treatment, that is to say at a time when the patient is no longer wearing an active orthodontic retainer apparatus.

The target model may therefore also be called the “initial” model.

Preferably, the target model is prepared less than six months, preferably less than three months, more preferably less than one month, after the end of the orthodontic treatment, generally immediately after the end of the treatment. It thus corresponds to a substantially optimal position of the teeth.

The form of the target model is not limited. It can in particular be a three-dimensional digital model, but also an image, a two-dimensional photo, for example a panoramic photo, or a film. In one embodiment, the target model may be obtained by one or more measurements carried out on the teeth, for example the measurement of a space between two adjacent teeth.

Preferably, the target model is a three-dimensional digital model of the patient's dentition, for example of the type .stl or .Obj, .DXF 3D, IGES, STEP, VDA or point clouds. Advantageously, such “3D” models may be observed at any desired angle.

The target model may be prepared from measurements carried out on the patient's teeth or on a physical model of his teeth, for example a plaster model.

The target model is preferably created by means of a professional apparatus 9, preferably in an orthodontic practice, for example by means of a 3D scanner, preferably used by a healthcare professional, for example by an orthodontist or an orthodontic laboratory 10. In an orthodontic practice, the patient 12 or the physical model of his teeth may advantageously be placed in a precise position and the acquisition means may be sophisticated. This results in a very precise target model.

The target model may be stored in a centralized database that includes the target models from a plurality of patients. This database may be physically set up in a specialized establishment. It may also be set up in a laboratory or an orthodontic practice, which limits the transfer of confidential information.

In one embodiment, the target model, or a copy of the target model, is given to the patient. Preferably, a computer file corresponding to the target model is stored on a portable medium, for example on a USB stick or on an electronic card, preferably on a cell phone, a tablet or a portable computer of the patient's, in particular on the personal apparatus that will be used at step b).

At step b), after a time interval Δt, an updated model 16 is created.

The time interval Δt may be predetermined. It may be constant, irrespective of the occurrence of the method, that is to say irrespective of whether this interval concerns the first execution of the method or a subsequent execution. It may be variable and depend, for example, on the results of an earlier step c). In particular, the time interval Δt may be shorter when this earlier step c) has detected a considerable deviation.

In a preferred embodiment, the time interval Δt is determined by the orthodontist according to a monitoring schedule. Depending on how the position of the teeth develops, the orthodontist is able to modify this schedule and thus modify the time interval Δt. In one embodiment, the monitoring method uses several cycles, each comprising a step b) and a step c), and the time intervals between each cycle may be identical or different. The time intervals between two successive cycles may all be determined before the first cycle is executed, in order to conform to a monitoring schedule planned by the orthodontist.

The time interval Δt may also be indeterminate and depend, for example, on decisions of the patient. For example, an updated model may be created during a visit to the dentist or whenever the patient wishes this to be done.

The time interval Δt is preferably determined to correspond to a potentially significant development in the position of the teeth. Preferably, for the first year after the treatment, the time interval Δt is less than three months. After this first year, the time interval Δt is preferably longer than one month, or even longer than six months or longer than twelve months. A time interval of between six months and eighteen months is suitable in particular for detecting a deviation of the teeth.

Preferably, the patient is sent at least one reminder informing the patient of the need for an updated model to be created. This reminder may be in paper form or, preferably, in electronic form, for example in the form of an e-mail, an automatic alert from the portable specialized app, or an SMS. A reminder of this kind may be sent by the orthodontic practice or laboratory 10, or by the dentist, for example.

The updated model may or may not be prepared like the target model. Its format may be identical to or different than that of the target model.

Preferably, the updated model 16 is created with a personal apparatus 18 currently available on the market, for example a cell phone or a tablet or a personal computer, either fixed or portable, preferably a photographic apparatus. The updated model 16 may in particular be created by the patient himself or by one of his relations. For this purpose, a specialized app is preferably loaded on the personal apparatus 18. The personal apparatus preferably weighs less than 3 kg, less than 2 kg, less than 1 kg, less than 500 g, preferably less than 300 g.

It is also preferable that the updated model 16 is produced from or is in the form of a photo, in particular a panoramic photo, or a film. In a preferred embodiment, the updated model is in the form of at least one photo, preferably at least three photos, corresponding to a view of the patient's teeth from the front, a view of the patient's teeth from the right and a view of the patient's teeth from the left. Photos may be taken either for the upper dental arch or for the lower dental arch or for both.

The personal apparatus preferably provides color images and/or infrared images of the patient's mouth or even of the patient's face. The infrared images advantageously show the teeth with excellent contrast.

As is shown in FIG. 2, the personal apparatus 18 has an acquisition or “capture” module 20 and preferably a processing module 22, also preferably a comparison module 24, and preferably a communication module 25.

Preferably, the patient's personal apparatus has a specialized app having one or more of these modules, preferably all of these modules. It is also preferable that this app organizes the reminders and informs the patient of the need to create an updated model.

Preferably, the specialized app is loaded onto the personal apparatus from a physical support such as a USB stick or a CD-ROM or is downloaded from the Internet or by radio. In one embodiment, the specialized app is made available to the patient by the orthodontic practice and/or the orthodontic laboratory. It may in particular take the form of an app of the kind currently downloaded on iPhones with the Apple® trademark, or apparatuses of all brands using Android® operating systems or any other operating system.

The acquisition module 20 preferably has a photographic apparatus or a video or infrared camera which the user, for example the patient or one of his relations, positions by means of a viewfinder or a screen before activating it.

Preferably, the acquisition module 20 has error prevention means that make this positioning easier.

The user can be guided in the acquisition by written messages and/or voice messages. For example, the personal apparatus may announce “Take a photo from the front”, emit a signal to inform the user that the photo is acceptable or, on the contrary, that he must take another photo, announce “Take a photo from the right”, preferably by displaying an arrow in order to orient the user, etc. The end of the acquisition process may also be announced by the apparatus. The apparatus may also help in the positioning of the personal apparatus, for example by visual messages (for example by display of arrows) and/or audio messages (such as a succession of beeps whose frequency increases as the positioning of the apparatus improves) and/or written messages and/or voice messages (“higher”, “lower”, etc.).

The error prevention means may in particular have locating marks which appear on the viewfinder or the screen. The locating marks may, for example, include a line intended to be aligned with the general direction of the join between the upper teeth and the lower teeth when the teeth are closed by the patient, or a vertical line intended to be aligned with the join between the two upper incisors. The locating marks may also refer to other parts of the patient. For example, they may consist of marks corresponding to the position of the eyes or may take the form of a contour within which the patient's mouth or face has to be positioned. In one embodiment, the locating marks correspond to a reference system, for example in the form of a retractor or an intraoral imaging device, which is preferably given to the patient at a visit to his orthodontist or dentist, and which the patient has to position in a predetermined position during the acquisition. For example, the reference system may be intended to be bitten by the patient.

In a preferred embodiment, the error prevention means are defined, at least in part, on the basis of information supplied by the target model. For example, in accordance with the principles of “augmented reality”, all or part of the target model, optionally reprocessed, may be rendered visible on said screen or said viewfinder during the acquisition.

The reprocessing of the target model in order to facilitate the acquisition, or “acquisition reprocessing”, may comprise, for example, the creation of one or more images or a three-dimensional view in which the teeth appear in transparency. It may also involve the generation of two-dimensional images, in particular from a three-dimensional target model, for example the generation of a front view, a view from the right and a view from the left.

The acquisition reprocessing may be carried out, for example, on a dedicated server, preferably by a dental professional, in particular immediately after the creation of the target model. Advantageously, the dental professional may have computing power available by which it is possible to obtain sophisticated error prevention means. The acquisition reprocessing is preferably carried out by the personal apparatus, in particular with the specialized app.

Preferably, the information deriving from the target model and used to create the error prevention means is downloaded to the personal apparatus 18.

Preferably, at least part of the target model appears in transparency to the user during the acquisition. In one embodiment, only the contour of the teeth appears to the user. It is therefore very easy for the latter to superpose the target model which appears in transparency with the teeth of the patient that he must take a photo of or film. In particular, when the creation of the updated model requires one or more photos to be taken, preferably photos from the front, from the right and from the left, the screen or the viewfinder preferably displays a corresponding view of the target model, preferably a line-type representation.

For this representation in transparency, the target model is preferably replaced by a reference model which is not specific to the patient, for instance which represents standard dental archs, like a typodont. The reference model can advantageously be used for a plurality of patients, which limits the need for data processing.

If the acquisition is made by video or by panoramic imaging, a real-time pre-rendering may be effected during the acquisition. This pre-rendering will guide the user on the missing zones and/or will select the photos or video images that are clearest or best interpolated to construct the updated model.

Preferably, the processing module 22 is configured to transform the information acquired by the acquisition model, for example photos, to an updated model that can be compared with the target model. The processing module 22 may, for example, process the acquired images in order to create a digital updated model, preferably in three dimensions.

Preferably, the processing module 22 also has means for refining the acquired information. For example, by comparing the acquired information and the information issuing from the target model, it is able to detect image deformations resulting from unsuitable positioning of the objective of the photographic apparatus, positioned at a low angle for example. In one embodiment, information from one or more previous updated models are used to refine the acquired information.

At step c), a comparison is made between the updated model and the target model.

Preliminary processing of the target model may be necessary for this purpose. For example, when the updated model preferably has a photo, the processing module 22 has means by which it is possible to identify, in the target model, the view presenting the greatest similarity to said photo. This view is then compared to said photo.

The comparison between the target model and the updated model may be carried out using two well-known algorithms:
Algorithms for comparison between 2D representations such as photos: These algorithms compare photos that are taken from the same viewpoint and/or that are deduced by analysis of a 3D model, especially in order to determine a view of this model corresponding to the desired orientation.
Algorithms for comparison between two three-dimensional models: A three-dimensional target model is then compared to a three-dimensional updated model, for example obtained by 3D extrapolation of 2D representations. This comparison may be carried out automatically or semi-automatically using what are known as 3D best-fit algorithms or manually using 3D alignment tools, especially by selecting, on each of the models, three points corresponding to identical sites. Alternatively, it is possible to calculate the difference in distance between characteristic points on the target model and the distance between these same characteristic points on the updated model. These characteristic points are preferably the cuspid points of the patient's teeth.

Preferably, the target model is loaded onto the personal apparatus by the party that created it, which limits the risks of disclosure of personal information. The comparison may then be made by the specialized app.

The comparison traditionally supplies a “distance” between the two models.

This distance may be interpreted directly by the patient, the orthodontist or the dentist. Preferably, the comparison module 24 compares said distance with an acceptance threshold and supplies practical information. In particular, if the distance is below the acceptance threshold, the practical information may be that no action is to be taken, whereas, if the distance is above the acceptance threshold, the practical information may be to the effect that a checkup visit to the dentist or orthodontist should be made.

In one embodiment, the comparison module supplies different practical information depending on the position of said distance with respect to several thresholds. For example, when the distance exceeds a critical threshold, the practical information may consist in conveying the need to consult the dentist or orthodontist as a matter of urgency.

The one or more thresholds used by the comparison module may be predetermined. In one embodiment, they may be parameterized. Preferably, they may be fixed and modified at any time, in particular by a dental professional.

Depending on the practical information received, the patient may visit the orthodontist or dentist in order to receive therapeutic or preventative treatment.

Preferably, the implementation of a step c) immediately brings about the establishment of a new time frame for carrying out new steps b) and c). Advantageously, the monitoring of the possible deviation of the teeth is thus permanent.

In one embodiment, the practical information is used to modify the time interval after which the patient will have to be alerted that a new updated model needs to be created.

In one embodiment, the individual apparatus makes it possible to display images, or even a sequence of images showing the position of the teeth at different dates. These images may be presented in the form of an animation.

In an embodiment, the cell phone makes it possible to display an image having first and second portions showing the positions of the teeth at different first and second dates, respectively, the first and second portion being preferably complementary so as to realistically represent the mouth of the patient.

Thereby, the patient visualizes his teeth, and preferably his mouth or his visage, as if a photo of his teeth, mouth or visage had been acquired. But depending on the portion of the image, he visualizes said teeth, mouth or visage at different dates.

Preferably, said first and second portions are separated by a line, preferably a vertical line. The first part, on the left side of the image, may represent the teeth in the target position, for instance at the beginning of an orthodontic treatment, and the second part, on the right side of the image, may represent the teeth in the updated position.

The first part, on the left side of the image, may represent the teeth in the updated position, and the second part, on the right side of the image, may represent the teeth in the target position, for instance at the end of an orthodontic treatment.

Preferably, the patient can interact with the cell phone so as to change the frontier between the first and second portions. Preferably, the screen is a tactile screen and the patient can move said vertical line by touching and moving the vertical line on the screen.

The communication module 25 is optional, particularly if, at step a), the target model and the specialized app have been loaded onto the personal apparatus.

It allows the personal apparatus 18 to receive the reminders intended for the patient, but also information that is useful for the other modules, for example information relating to the target model, especially in order to create locating marks. The communication module 25 also makes it possible, if need be, to transmit the updated model and/or the results of the comparison made at step c), and in particular practical information, especially to a dentist or to an orthodontist.

Said information may be transmitted immediately or at predetermined moments. For example, the updated models may be sent to the orthodontist, especially in the form of reports, at a parameterizable frequency.

The communication module 25 is preferably configured to transmit and/or receive data in a secure manner.

For example, the communication may be performed, at least in part, by radio, preferably using at least one protocol chosen from among the protocols edge, 3G, 4G, udmsa, hpdmsa, Bluetooth and Wi-Fi, or using any other protocol, current or future, that is suitable for mobile or nomadic equipment, by wired synchronization with the personal computer, or by optical transmission.

As is clear at present, a method according to the invention permits precise and effective monitoring of developments in the position of the patient's teeth, substantially without any stress for the patient. The patient may thus easily implement this method, and the risk of relapse is thus greatly reduced.

This method is not in any way involved in maintaining the teeth in their position at the end of treatment. It does not replace the fitting of a retainer at the end of treatment.

Of course, the invention is not limited to the embodiments that have been described above and that are illustrated.

Preferably, the processing of the acquired images and the comparison are carried out by the same personal apparatus as the one that performs the acquisition. It is also preferable that the target model is loaded onto this personal apparatus, preferably by the orthodontic practice or the orthodontic laboratory that created the target model. Advantageously, the communication of confidential data is thereby limited.

However, several different apparatuses may also be used. For example, the acquisition may be performed with a cell phone, and the processing and comparison by a fixed computer.

Furthermore, the processing of the captured images and/or the comparison are not necessarily implemented in the personal apparatus. In particular, one or more of these modules may be implemented in a dental practice, an orthodontic practice or an orthodontic laboratory. For example, in one embodiment, step b) may be implemented by the dentist or the orthodontist.

Thus, all of the features that relate to the personal apparatus and that have been described above may be applied to the apparatus used by the dentist or the orthodontist.

Acquisition by the dentist or the orthodontist advantageously permits good positioning of the patient, which ensures precise images and improves the quality of the comparison.

In addition, the safety of the transfer of information relating to the target model or to the updated model (especially if the comparison is made outside the premises of the dentist or of the orthodontist) is advantageously improved in this way.

Preferably, the acquisition module and/or the processing module and/or the comparison module are then integrated in the professional equipment of the dentist or orthodontist. It is also preferable that the acquisition is performed in predetermined positions, preferably automatically, that is to say without the intervention of the dentist or orthodontist.

For the dentist, the implementation of the method advantageously makes it possible to detect shifts that are imperceptible to the eye, for example a loss of expansion, or shifting of the teeth with respect to a target model which does not correspond to an ideal position but, for example, the best position that the orthodontic treatment has been able to produce.

Moreover, the field of application of a method according to the invention is not limited to the detection of a deviation in the position of the teeth in order, subsequently, to take measures to counteract this deviation.

A method according to the invention may also be used to monitor the development of the position of the teeth independently of an orthodontic treatment, for example to check whether the teeth do not move towards prejudicial positions or reach such prejudicial positions, and/or to measure the speed at which the teeth move. Such a measurement may be useful for study purposes.

By repeating steps b) and c), it is also possible to evaluate the speed at which an orthodontic treatment causes the position of the teeth to change, and thereby to measure the efficacy of this treatment. In this embodiment, step a) is optional.

A method according to the invention may be used, for example, for remote monitoring of the course of an orthodontic treatment, thereby optimizing visits by patients to their orthodontists.

The target model does not necessarily correspond to an optimal position of the teeth. A method according to the invention may thus be used in the context of an orthodontic treatment. In particular, the target model may be a theoretical model that the orthodontic treatment seeks to attain, at the end of the orthodontic treatment or at an intermediary instant between the beginning and the end of the orthodontic treatment. For instance, the target model may be a 3D model or a 2D model, preferably a 3D model, that is expected to be reached at a determined intermediary instant. By implementing the method, the patient is thus able, when he so desires, to evaluate the change in position of his teeth and assess the approximation to the theoretical model.

Said 2D or 3D model may be created before the beginning of the orthodontic treatment. In particular, an orthodontic treatment with aligners conventionally requires the generation of a plurality of 3D models of the teeth to design a plurality of corresponding aligners. The 3D model may be one of these models.

A method according to the invention may also be used to monitor the shape and/or the color of the patient's teeth. More generally, it may be used to monitor the position, and/or the shape and/or the color of any element in the mouth, and in particular a tooth, but also a soft tissue, and in particular the gingiva, and/or an orthodontic part or a dental part such as a bracket, an archwire or an aligner, or a dental part such as a filling, a dental veneer, an implant or a dental prosthesis.

The invention therefore concerns a method for monitoring the position, the shape, or the color of an element in the patient's mouth, said method comprising the following steps:
a) modeling a target position, shape, or color of said element, in the form of a target model, preferably a 3D model, preferably generated with a 3D scanner, or an image, preferably a photo, preferably generated with a cell phone;
b) after a time interval, preferably longer than one week, or longer than one month, modeling an updated position, shape, or color of said element, respectively, in the form of an updated model;
c) comparing said target and updated models,
method in which the updated model is produced from, or is in the form of, a photo or a film, said photo or film being acquired with a cell phone by the patient himself or by one of his relations, preferably in a location which is not a dental site, for instance in a location which is more than 500 m or more than 1 km away from a dental site, and in particular away from a dental office or from an orthodontist office.

Advantageously, the invention may be used to monitor
a change in the position of a tooth,
a change of shape of a tooth resulting in particular from the wear of the tooth, for instance in case of a bruxism, or resulting from the growth of the tooth or from the detachment of the tooth, or from the detachment or the addition of a filling,
a change of shape of a gingiva resulting in particular from a swell of the gingiva, for instance in case of a gingivitis, or from a retraction of the gingiva (in case of recession),
a change of shape of an orthodontic part, for instance resulting from a failure, an unseat, or a detachment of an orthodontic part, for instance of an archwire or of a bracket of an orthodontic appliance, a loss of tie or clip of brackets opened or damaged,
a change of oral hygiene of the patient,
a change of shape of a dental part, for instance resulting from a failure of a filling,
a change of color resulting from a stain, or from a whitening operation, or from the ingestion of a coloring ingredient, for instance coffee or tea, or from smoking, in particular smoking of cigarettes or cigars.

This monitoring may in particular be used to assess the patient compliance to a dental or orthodontic treatment and/or to measure the efficiency of a dental or orthodontic treatment.

The features of the method which are disclosed hereabove to monitor the position of teeth may be applied to monitor the shape or the color of any element in the patient's mouth.

At step a), preferably, the target model is an image or is created by means of a 3D scanner.

In an embodiment, the target model is an image, preferably a photo, preferably acquired by the patient or by one of his relations with the cell phone, for instance an updated model of a previous execution of the method of the invention.

Preferably, before step b), for instance less than 1 month, preferably less than 1 week before step b), the cell phone informs the patient of the need for an updated model to be created.

At step b), the patient may take at least one photo, for instance at home, with his cell phone. This at least one photo may be the updated model, or may be processed, by said cell phone, or preferably by a computer in a remote central computer to which the cell phone sends said at least one photo, to get an updated model comparable to the target model, or easier to compare to the target model.

In a preferred embodiment, the target model is a view of a 3D model (i.e. an observation in two dimensions of the 3D model), preferably of a 3D model acquired with a 3D scanner, or an image resulting from a processing of said view. The updated model is preferably a photo taken by the patient or by one of his relations with a cell phone, or an image resulting from a processing of said photo.

The image processing methods are well-known. For instance, they include methods to improve the contrast or the sharpness of the image (for instance filtering methods) or to modify the dimensions of the image (for instance cropping methods), to isolate an element of the image, to specifically process this element. Processing methods include for instance classification algorithms, in particular with a neuronal network, in particular a CNN neuronal network, or preferably a VGG neuronal network with a SE.Detection block («squeeze-and-excitation (SE)» blocks are in particular disclosed by Jie Hu et al, in «Squeeze-and-Excitation Networks», arXiv:1709.01507v4 [cs.CV] 16 May 2019).

The processing may also combine the different photos, for instance to create a 3D model as an updated model.

A step c), said algorithms for comparison between 2D representations such as photos or for comparison between two three-dimensional models may be used.

Preferably, depending on the comparison at step c), the patient and/or a dental professional is informed of the probable need for a consultation with a dentist or an orthodontist, and/or a visit to an orthodontist or a dentist is scheduled.

Step c) is preferably achieved by said central computer, which sends the result of said comparison to said cell phone.

Preferably, said central computer
has access to a plurality of target models related to respective patients, preferably related to more than 1,000, preferably more than 10,000 patients, and/or less than 100,000 patients;
receives the updated models from a plurality of cells phones belonging to said patients;
compares said target and updated models;
sends the result of said comparison to said cell phones.

Finally, the patient is not limited to a human being. In particular, a method according to the invention may be used for another animal.

1. A method for monitoring the position of a patient's teeth, said method comprising the following steps:
a) modeling a target position of said teeth in the form of a target model;
b) after a time interval, modeling an updated position of said teeth in the form of an updated model;
c) comparing said target and updated models,
the updated model being created by the patient himself or by one of his relations.

2. The method as claimed in claim 1, in which the updated model is produced from photos or from a film acquired with a cell phone.

3. The method as claimed in claim 2, in which the updated model is in the form of at least one photo or a film.

4. The method as claimed in claim 2, in which said photos and film are processed in order to create a digital updated model in three dimensions.

5. The method as claimed in claim 2, in which the updated model is produced from at least three photos, corresponding to a view of the patient's teeth from the front, a view of the patient's teeth from the right and a view of the patient's teeth from the left.

6. The method as claimed in claim 3, in which the updated model is in the form of at least at least three photos, corresponding to a view of the patient's teeth from the front, a view of the patient's teeth from the right and a view of the patient's teeth from the left.

7. The method as claimed in claim 2, in which the time interval between steps a) and b) is indeterminate and depends on decisions of the patient.

8. The method as claimed in claim 2, in which the time interval between steps a) and b) is determined by the orthodontist according to a monitoring schedule and/or his decision

9. The method as claimed in claim 2, in which, before step b), the patient is sent at least one reminder informing him of the need for an updated model to be created.

10. The method as claimed in claim 2, in which a specialized app is downloaded onto the cell phone from the Internet or by radio, and comprises an acquisition module having error prevention means to guide the patient during step b).

11. The method as claimed in claim 10, in which the error prevention means guide the patient or said one of his relations by written messages and/or voice messages and/or on the basis of information supplied by the target model.

12. The method as claimed in claim 10, in which at least part of the target model or at least a part of a reference model appears in transparency to the patient or to said one of his relations, during said acquisition.

13. The method as claimed in claim 2, in which the target model is an image.

14. The method as claimed in claim 13, in which the target model is a photo or a view of a 3D model.

15. The method as claimed in claim 3, in which the target model is an image.

16. The method as claimed in claim 15, in which the target model is a photo or a view of a 3D model.

17. The method as claimed in claim 2, in which the target model is a three-dimensional digital model.

18. The method as claimed in claim 3, in which the target model is a three-dimensional digital model.

19. The method as claimed in claim 2, in which
the target model is a three-dimensional digital model, and
photos are acquired, at step b), with a cell phone, and processed in order to create a digital updated model in three dimensions.

20. The method as claimed in claim 19, in which, at step c), the comparison is carried out automatically or semi-automatically using a 3D best-fit algorithm.

21. The method as claimed in claim 2, in which, at step b), one or more photos and/or videos of the patient's teeth are processed in order to create a three-dimensional updated model.

22. The method as claimed in claim 2, in which the target model is a three-dimensional digital model, and the updated model is a three-dimensional updated model obtained from 2D representations.

23. The method as claimed in claim 2, in which, at step c), said comparison supplies a distance between the target model and the updated model, and practical information is supplied, said practical information depending on a comparison between said distance and an acceptance threshold.

24. The method as claimed in claim 23, in which different practical information is supplied, depending on a position of said distance with respect to several thresholds fixed by a dental professional.

25. The method as claimed in claim 23, in which, depending on the comparison at step c), the patient and/or a dental professional is informed of the probable need for a consultation with a dentist or an orthodontist, and/or a visit to a dental site, preferably to an orthodontist or a dentist, is scheduled, and/or is informed that an orthodontic appliance worn by the patient needs to be corrected or replaced.

26. The method as claimed in claim 2, in which the cell phone makes it possible to display images, or a sequence of images showing the position of the teeth at different dates.

27. The method as claimed in claim 26, in which the cell phone makes it possible to display an image having first and second portions showing the positions of the teeth at different first and second dates, respectively, the first and second portion being preferably complementary so as to realistically represent the mouth of the patient, said first and second portions being preferably separated by a vertical line which, preferably may be moved by an interaction with the screen.

28. The method as claimed in claim 2, in which steps b) and c) are repeated to evaluate the speed at which an orthodontic treatment causes the position of the teeth to change.

29. The method as claimed in claim 2, in which, at step b), in accordance with principles of “augmented reality” or in transparency, all or part of the target model, optionally reprocessed, is rendered visible on a screen of a cell phone operated by the patient or by said one of his relations.

30. The method as claimed in claim 29, in which the target model reprocessing comprises the creation of one or more images or a three-dimensional view in which the teeth appear in transparency, and/or involves a generation of two-dimensional images.

31. The method as claimed in claim 30, in which the target model is a three-dimensional target model, and said two-dimensional images are images from said three-dimensional target model.

32. The method as claimed in claim 31, in which the screen displays a view of the target model corresponding to a photo to be taken with said cell phone.

33. The method as claimed in claim 2 further comprising using a result of the comparing to evaluate the efficacy of an orthodontic treatment, and/or evaluate the change of position of teeth toward a theoretical model corresponding to a defined position of the teeth.

34. The method as claimed in claim 2 further comprising using a result of the comparing to monitor the development of the position of teeth independently of an orthodontic treatment.

35. The method as claimed in claim 2 further comprising remotely monitoring a course of an orthodontic treatment.

36. A method for monitoring a position, a shape, or a color of an element in a patient's mouth, said method comprising the following steps:
a) modeling a target position, shape, or color of said element, in the form of a target model;
b) after a time interval longer than one month, modeling an updated position, shape, or color of said element, respectively, in the form of an updated model;
c) comparing said target and updated models,
in which the updated model is produced from, or is in the form of, a photo or a film, said photo or film being acquired with a cell phone by the patient himself or by one of his relations in a location which is not a dental site.

37. The method as claimed in claim 37, in which the element is chosen in the group consisting of a tooth, a soft tissue, a dental part and an orthodontic part.

38. The method as claimed in claim 37, in which, depending on the comparison at step c), the patient and/or a dental professional is informed of the probable need for a consultation with a dentist or an orthodontist, and/or a visit to an orthodontist or a dentist is scheduled.

39. The method as claimed in claim 37, in which the target model is an image or is created by means of a 3D scanner.

40. The method as claimed in claim 37, in which, prior to step a), the cell phone informs the patient of the need for an updated model to be created.

41. The method as claimed in claim 37 further comprising using a result of the comparing to monitor
a change in the position of a tooth,
a change of shape of a tooth resulting from a wear of the tooth, from a growth of the tooth, from a detachment of the tooth from a dental arch of the patient, or from an addition of a filling in the tooth,
a change of shape of a gingiva resulting from a swell of the gingiva or from a retraction of the gingiva,
a change of oral hygiene of the patient,
a change of shape of a dental archwire or of a bracket,
a change of color resulting from a stain, from a whitening operation, from an ingestion of a coloring ingredient, or from smoking, and/or
to assess the patient compliance to a dental or orthodontic treatment, and/or
to measure the efficiency of a dental treatment or of an orthodontic treatment.
